(19) United States
(12) Patent Application Publication
(10) Pub. No.: US 2013/0012992 A1
(43) Pub. Date: Jan. 10, 2013

(54) APPARATUS FOR COMPRESSING OR DECOMPRESSING A SPINAL DISC AND METHOD OF USE THEREOF

(76) Inventors: NASSER ANI, Holmdel, NJ (US); DARREN FREEMAN, Morganville, NJ (US)

(21) Appl. No.: 13/616,821

(22) Filed: Sep. 14, 2012

Related U.S. Application Data
(63) Continuation-in-part of application No. 12/605,402, filed on Oct. 26, 2009.

(51) Int. Cl. A61B 17/70

(52) U.S. Cl. 606/246

(57) ABSTRACT

An apparatus for compressing or decompressing a spinal disc comprises; a first section and a second section, wherein the first section defines a through hole configured to receive a fastener for coupling the first section to a first vertebrae, and wherein the second section defines a through hole configured to receive a fastener for coupling the second section to a second vertebrae; and an expandable section coupled to the first section and the second section for adjusting a distance between the first and second section.

### CROSS-REFERENCE TO RELATED APPLICATIONS

This application is a continuation-in-part of co-pending U.S. patent application Ser. No. 12/605,402, filed Oct. 26, 2009, and entitled, “APPARATUS FOR COMPRESSING OR DECOMPRESSING A SPINAL DISC AND METHOD OF USE THEREOF,” which is herein incorporated by reference in its entirety.

### BACKGROUND OF THE INVENTION

1. Field of the Invention

Embodiments of the present invention generally relate to medical devices and, more particularly, to an apparatus for decompressing a spinal disc and method of use thereof.

2. Description of the Related Art

A spine generally consists of the vertebral column having a plurality of vertebrae linearly stacked atop one another, protecting the spinal cord and providing support for the upper body. Between the vertebrae are discs that cushion the vertebrae and promote smooth movement of the vertebral column.

Due to age, use, or physical trauma, the discs may become damaged. In some instances the outer wall of the disc (annular fibrosis) may become weakened and tear, causing the soft inner part of the disc (nucleus pulposus) to protrude out of the disc. This condition is commonly known as a herniated, compressed, slipped, or bulged disc. Symptoms of a damaged disc may range from mild to severe. A patient having a damaged disc may experience pain, soreness, numbness, weakness of muscles, nerve damage and even partial paralysis. In addition, secondary complications may occur. For example, a narrowing of the spinal canal (spinal stenosis) or the narrowing of the lateral openings of the spinal canal (foraminal stenosis) may occur, which may result in undue pressure being placed on the nerves.

Treatments typically begin with non-surgical solutions, such as physical therapy, pain management, steroid injections, or rest. However, in the event that non-surgical treatments do not successfully alleviate the effects of a damaged disc, surgery is required. Various techniques have been employed to correct a damaged disc. For example, one commonly used technique includes removing portions of the bone from the vertebrae (e.g., laminectomy) to relieve pressure on the spinal column. However, as with any procedure that involves the removal of bone, post surgery recovery may involve a significant amount of pain for extended periods of time. In addition, removing portions of the vertebrae may create instability of the spine. In such instances an implant may be inserted between or anchored to the vertebrae to stabilize the spine. However, such implants effectively fuse the vertebrae together, creating a lack of flexibility, thus causing a permanent decrease of mobility for the patient following the surgery.

Other techniques involve removing part of the damaged disc (e.g., cervical or lumbar disectomy). However, while temporary relief is attained, such techniques do not guarantee a damaged disc will heal or prevent further degeneration. To achieve more permanent results, similar techniques include completely removing the damaged disc and replacing it with a synthetic disc or a hinged implant. However, over time the synthetic disc or hinged implant may eventually degenerate, requiring additional surgeries to replace the worn out parts.

Therefore, there is a need in the art for an improved apparatus and method for performing corrective spinal surgery.

### SUMMARY

An apparatus for compressing or decompressing a spinal disc is provided herein. In some embodiments, an apparatus for compressing or decompressing a spinal disc comprises; a first section and a second section, wherein the first section defines a through hole configured to receive a fastener for coupling the first section to a first vertebrae, and wherein the second section defines a through hole configured to receive a fastener for coupling the second section to a second vertebrae; and an expandable section coupled to the first section and the second section for adjusting a distance between the first and second section.

Other and further embodiments of the present invention are described below.

### BRIEF DESCRIPTION OF THE DRAWINGS

Embodiments of the present invention, briefly summarized above and discussed in greater detail below, can be understood by reference to the illustrative embodiments of the invention depicted in the appended drawings. It is to be noted, however, that the appended figures illustrate only exemplary embodiments of this invention and are therefore not to be considered limiting of its scope, for the invention may admit to other equally effective embodiments.

FIG. 1 depicts a perspective view of an apparatus for compressing or decompressing a spinal disc in use in accordance with some embodiments of the present invention.

FIG. 1A depicts a cross sectional view along line 1A-1A of the apparatus for compressing or decompressing a spinal disc of FIG. 1.

FIG. 2 depicts a perspective view of an apparatus for compressing or decompressing a spinal disc in accordance with some embodiments of the present invention.

FIG. 3 depicts a front view of an apparatus for compressing or decompressing a spinal disc in accordance with some embodiments of the present invention.

FIG. 4 depicts a cross sectional view along line 4-4 of the apparatus for compressing or decompressing a spinal disc of FIG. 3.

FIG. 5 depicts a flow diagram of a method for compressing or decompressing a spinal disc in accordance with some embodiments of the present invention.

FIGS. 6A-H are illustrative perspective views of a segment of a vertebral column during different stages of the method for compressing or decompressing a spinal disc depicted in FIG. 5, in accordance with some embodiments of the present invention.

FIG. 7 depicts a perspective view of an apparatus for compressing or decompressing a spinal disc in accordance with some embodiments of the present invention.

FIG. 8 depicts a cross sectional view along line 8-8 of the apparatus for compressing or decompressing a spinal disc of FIG. 7.

FIG. 9 depicts a front view of the apparatus for compressing or decompressing a spinal disc in accordance with some embodiments of the present invention.

To facilitate understanding, identical reference numerals have been used, where possible, to designate identical elements that are common to the figures. The figures are not drawn to scale and may be simplified for clarity. It is contemplated that elements and features of one embodiment may be beneficially incorporated in other embodiments without further recitation.

### DETAILED DESCRIPTION

Embodiments of the present invention generally relate to medical devices. The inventive apparatus and method includes an apparatus for compressing or decompressing a spinal disc and method of use thereof. The inventive apparatus and method advantageously provides a minimally invasive means for correcting a damaged spinal disc, allowing for a reduced post-surgery recovery time and increased post-surgery mobility for patients undergoing the procedure.

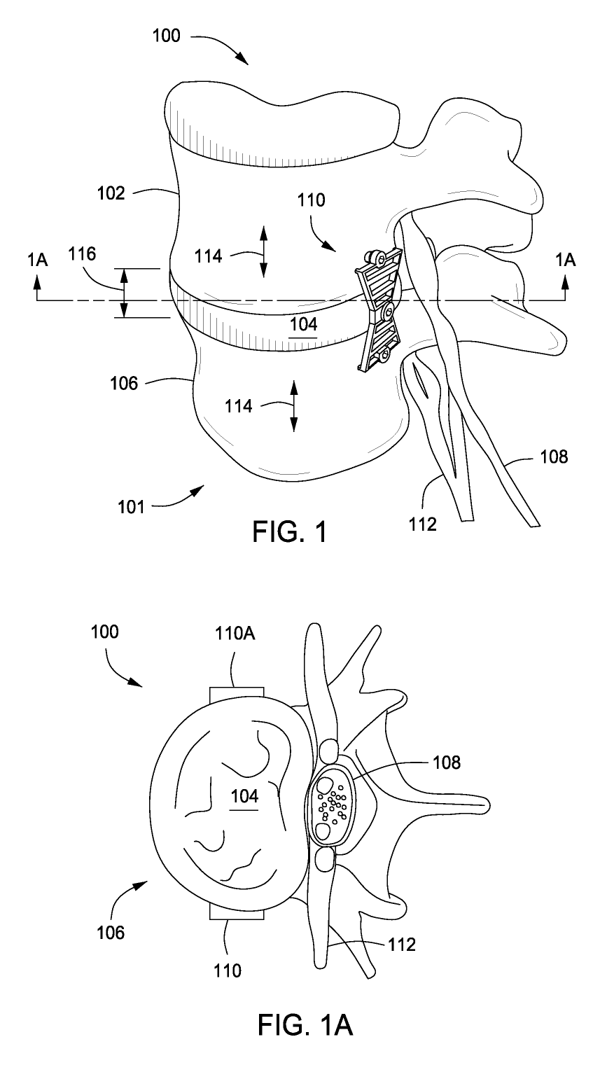

FIG. 1 depicts a perspective view of an apparatus for compressing or decompressing a spinal disc in use in accordance with some embodiments of the present invention.

A spine (segment shown) 100 generally comprises a vertebral column 101 consisting of a plurality vertebrae (two shown) 102, 106 linearly stacked atop one another, protecting the spinal cord or the neural elements 112. Attached to the spinal cord or the neural elements 112 are a plurality of nerves (one shown) 108 that extend throughout the body. Between the vertebrae 102,106 are spinal discs (one shown) 104 that generally comprise an inner gel-like substance (nucleus pulposus) surrounded by strong annular fibers (annulus fibrosus). The spinal disc 104 acts as a cushion between adjacent vertebrae 102, 106 to absorb forces exerted during movement of the vertebral column 101 and allows for smooth movement of the vertebrae 102, 106.

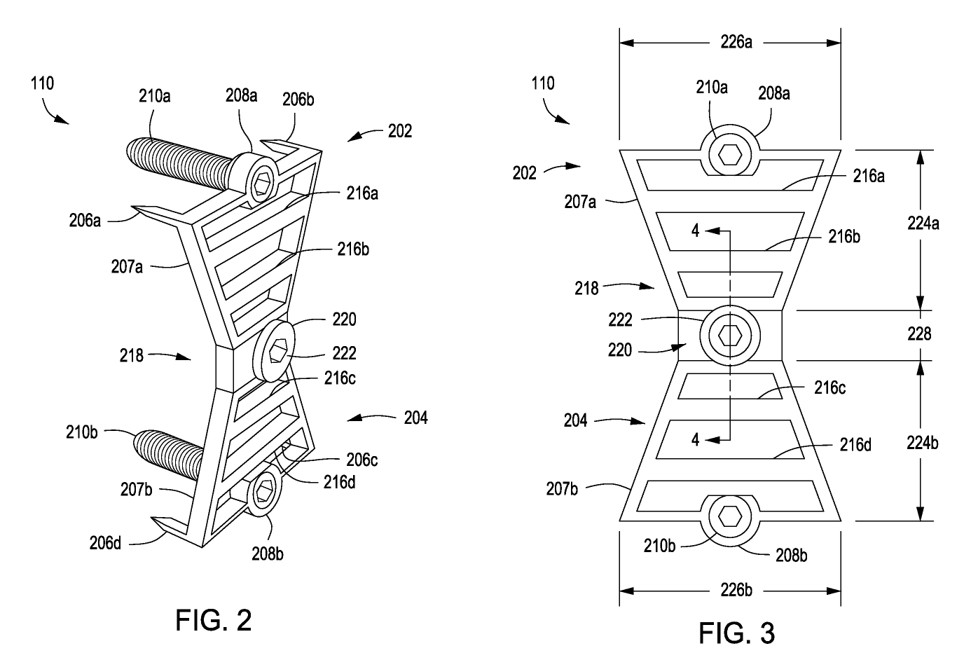

The disc apparatus 110, described more fully below with respect to FIGS. 2-5, may be simultaneously coupled to one or more surfaces of two adjacent vertebrae 102, 106 and configured to provide a force in a vertical direction 114, facilitating control of a distance (arrow 116) between the vertebrae 102, 106. For example, a force may be applied in a vertical direction 114 to increase the distance between the vertebrae 102, 106, thereby alleviating pressure and providing decompression of the spinal disc 104. Alternatively, the force may be decreased to decrease the distance between the vertebrae 102, 106, thereby applying pressure to, and providing compression of, the spinal disc.

The disc apparatus 110 may be positioned in any location suitable to provide stability to the vertebral column and facilitate compression or decompression of the spinal disc 104. For example, in some embodiments, such as depicted in FIG. 1, one disc apparatus 110 may be positioned to provide stability and facilitate compression or decompression of the spinal disc 104 on one side of the vertebral column 101. Alternatively, in some embodiments, such as depicted in FIG. 1A, a cross sectional view along line 1A-1A of FIG. 1, two or more disc decompression apparatus (two shown) 110, 110A may be positioned on opposing sides of the vertebral column 101. In such embodiments, both disc decompression apparatus 110, 110A may provide an equal and symmetrical force on opposing sides of the vertebral column 101, thereby promoting increased stability. Alternatively, each disc apparatus 110, 110A may provide an opposite or unequal force on opposing sides of the vertebral column 101, thereby providing compression of the spinal disc at one location of the vertebral column 101 and decompression of the spinal disc at another location of the vertebral column 101.

The placement of the disc apparatus 110 may vary to correct deformities or alleviate symptoms associated with a specific condition presented. For example, in embodiments where the disc apparatus 110 is being utilized to treat a herniated spinal disc (e.g. paracentral, central, bilateral or unilateral), one or more disc apparatus 110 may be positioned in a location suitable to provide selective compression or decompression in the location where the herniation occurred. In addition, the disc apparatus 110 may be used in conjunction with other surgical procedures (e.g. physical repair or replacement of a damaged disc) to assist in the healing process. For example, the disc apparatus 110 may be positioned approximate the location of a repaired area of the spinal disc 104 to provide support to the spinal disc 104 while the repaired area heals.

In some embodiments, the disc apparatus 110 may be utilized as a preventive measure. For example, the disc apparatus 110 placed above or below an existing damaged disc (pre-surgery or post-surgery) to avoid early degeneration of an adjacent damaged disc. Similarly, in some embodiments, the disc apparatus 110 may be placed at various locations along the spine 100 to provide support to an unnaturally curved spine (e.g. scoliosis) and prevent further damage as a result of the condition.

Referring to FIG. 2, the disc apparatus 110 generally comprises a first section 202, second section 204, and an expandable section 218.

The first section 202 and second section 204 may comprise any material suitable to provide sufficient mechanical strength and stiffness to the disc apparatus 110 while having a low overall weight. For example, the first section 202 and second section 204 may comprise a metal, plastic, ceramic, or the like. In some embodiments, the metal may be at least one of titanium, steel, aluminum, alloys thereof, or the like. In some embodiments, the material comprises a tensile strength sufficient to resist permanent deformation under the stresses applied thereon resulting from the natural movement of the vertebral column. In some embodiments, the tensile strength may be between about 200 to about 1000 MPa. For example, in embodiments where the material comprises a stainless steel, the tensile strength may be between about 200 to about 225 MPa. Alternatively, in embodiments where the material comprises an aluminum alloy, the tensile strength may be between about 800 to about 925 MPa. The material may also comprise one or more other beneficial properties, for example, resistance to corrosion, pitting, abrasion, chemical degradation, and the like.

The first section 202 and second section 204 may comprise any shape suitable to provide an adequate surface area to facilitate a stable coupling of the disc apparatus 110 to a vertebral column and provide sufficient mechanical stiffness, while not impeding movement. For example, the first section 202 and second section 204 may be square, triangular, circular, or the like. In some embodiments, such as depicted in FIG. 2, the first section 202 and second section 204 may be trapezoidal. The first section 202 and second section 204 may be the same shape, or in some embodiments, may comprise different shapes.

In some embodiments, the first section 202 and second section 204 may comprise a solid plate. Alternatively, in some embodiments, such as depicted in FIG. 2, the first section 202 and second section 204 comprise a trapezoidal frame 207a, 207b having a plurality of cross members 216 a-d to reduce the overall weight of the disc apparatus 110 while maintaining adequate strength.

In some embodiments, the first section 202 and second section 204 further define one or more through holes (two shown) 208a, 208b configured to receive a fastener 210a, 210b to facilitate coupling the disc apparatus 110 to a vertebral column. The fastener 210a, 210b may be any fastener suitable to provide a secure and permanent coupling. For example, the fastener may be a screw, bolt, anchor, or the like. In embodiments where the fastener 210a, 210b is threaded (e.g. screw or bolt) the through hole 208a, 208b may comprise threads configured to interface with threads of the fastener 210a, 210b used. The fastener 210a, 210b may be any length suitable to provide a secure coupling, while not damaging the structural integrity of the vertebrae. For example, in some embodiments, the fastener 210a, 210b may be between about 2.0 to about 4.0 cm long, or in some embodiments, about 2.5 to about 3.5 cm long.

In some embodiments, the first section 202 and second section 204 further comprise one or more (four shown, two on each section) posts 206 a-d to facilitate securing the disc apparatus 110 in a static position when coupled to a vertebral column. The posts 206 a-d may comprise a pointed or sharpened end to allow for the posts 206 a-d to breach the surface of the vertebral column. The posts may be any length suitable to facilitate a secure coupling of the disc apparatus 110 in a static position. For example, the posts 206 a-d may be about 0.1 to about 1.5 cm in length.

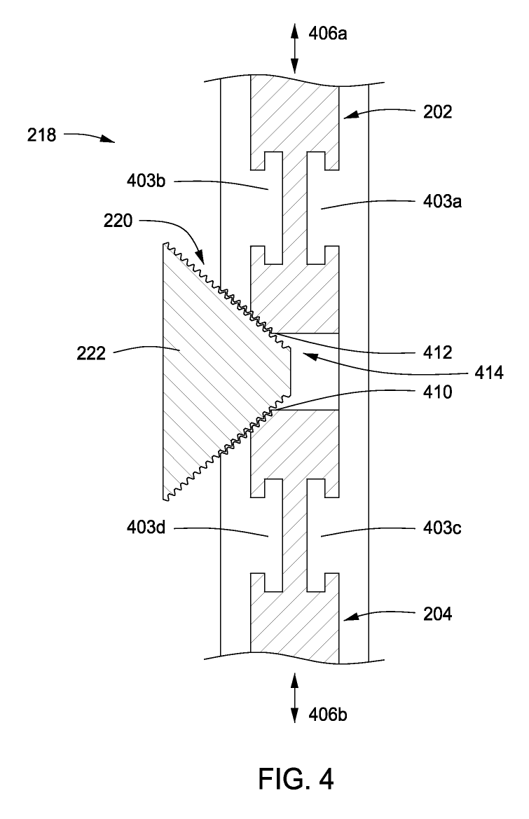

The expandable section 218 couples the first section 202 to the second section 204. In some embodiments, for example in embodiments described below with respect to FIG. 4, the expandable section 218 generally comprises a flexible, resilient material. The flexible, resilient material may comprise any material suitable to provide flexibility and expansion of the expandable section 218, while retaining some stiffness as to not buckle under forces exerted thereon. For example, the material may be an elastomer, such as rubber. Proximate the center of the expandable section 218 is a hole 220 configured to receive a screw 222. Alternatively or in combination, in some embodiments, the expandable section 218 may comprise a mechanical apparatus (not shown) to facilitate expansion of the expandable section 218. For example, the mechanical apparatus may include a plurality of movably coupled members configured to allow for precise expansion of the expandable section 218, such as a scissor jack. The mechanical apparatus may also comprise other mechanical elements, such as springs, mechanical slides, linear actuators, a combination thereof, or the like.

Referring to FIG. 3, in some embodiments the disc apparatus 110 may comprise any dimensions sufficient to securely couple each of the first section 202 and second section 204 to a respective vertebra and provide support to the vertebral column and facilitate decompression of a damaged spinal disc while not impeding movement or only partially restricting movement thereof. Additionally, the dimensions of the disc apparatus 110 may be varied to accommodate the various sizes of vertebrae with respect positions of the spinal column (e.g., the lumber, thoracic and cervical regions). For example, in some embodiments, the height (arrows 224a, 224b) of the first section 202 and second section 204 may be between about 0.4 to about 4.0 cm. In some embodiments, the width (arrows 226a, 226b) of the respective terminal ends 203, 205 of the first section 202 and second section 204 may be between about 0.4 to about 6.0 cm. The expandable section 218 may comprise any height (arrow 228) suitable to allow for proper placement of the disc apparatus 110 with respect to two adjacent vertebrae, while also comprising an adequate height when expanded to provide a sufficient spacing between the two adjacent vertebrae to facilitate a decompression of a damaged spinal disc. For example, the expandable section 218 may have a height of between 0.4 to about 2.0 cm and expand an additional about 0.3 to about 1.5 cm.

Referring to FIG. 4, a cross sectional view along line 4-4 of the disc apparatus 110 of FIG. 3, the first section 202 and second section 204 may be coupled to the expandable section 218 via any means suitable to facilitate a permanent coupling of the first section 202 and second section 204 to the expandable section 218. For example, the first section 202 and second section 204 may be coupled to the expandable section 218 via a binder, such as glue, cement, epoxy, or the like. Alternatively, in some embodiments, such as depicted in FIG. 4, the first section 202 and second section 204 may be coupled to the expandable section 218 mechanically, such as via a plurality of “T”—shaped features 403 a-d.

In some embodiments, such as depicted in FIG. 4, the hole 220 and screw 222 may comprise corresponding tapered threads. In such embodiments, the terminal ends 412, 410 of the first section 202 and second section 204 may also comprise a series of tapered threads 414.

In operation, the screw 222 is rotated to increase or decrease the distance between the first section 202 and second section 204. To increase the distance between the first section 202 and second section 204 the screw 222 is rotated and driven in a lateral direction into the expandable section 218. As the screw 222 progresses deeper into the hole 220 of the expandable section 218, a force is applied simultaneously to the first section 202 and second section 204 in a vertical direction 406a, 406b, increasing the distance between the first section 202 and second section 204, thereby causing the expandable section 218 to expand. To decrease the distance between the first section 202 and second section 204 the screw 222 is rotated out of the expandable section 218. As the screw 222 retreats from the hole 220 of the expandable section 218, the force applied to the first section 202 and second section 204 is decreased, thereby causing the expandable section 218 to contract.

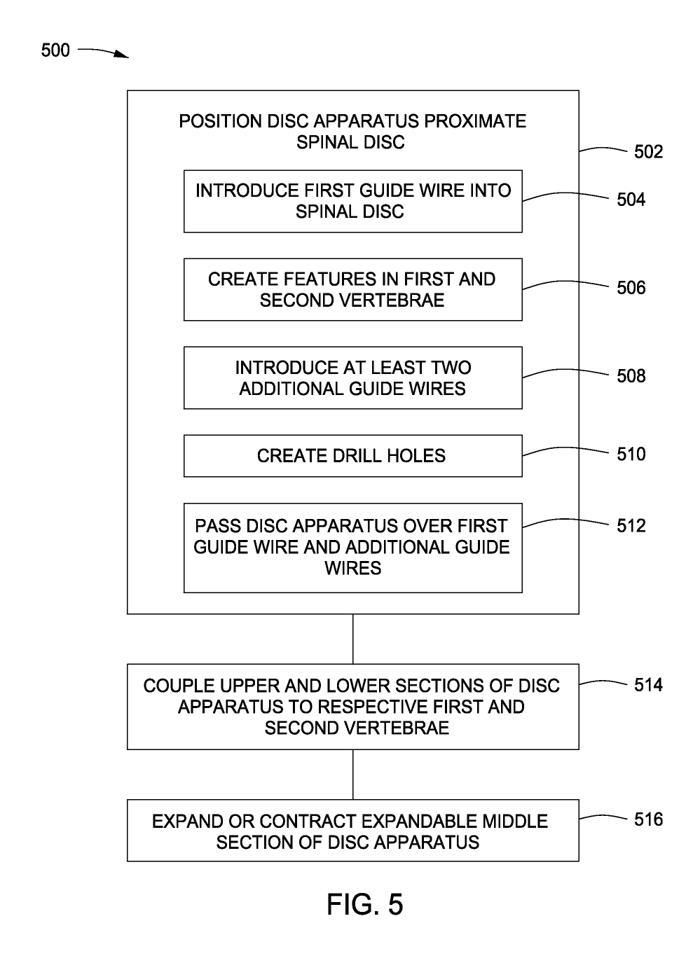

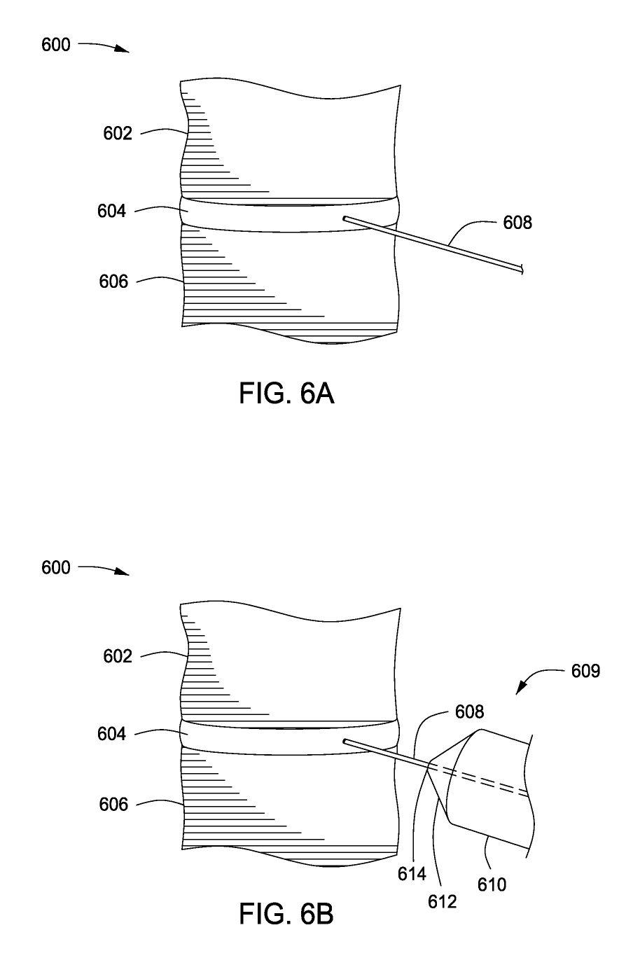

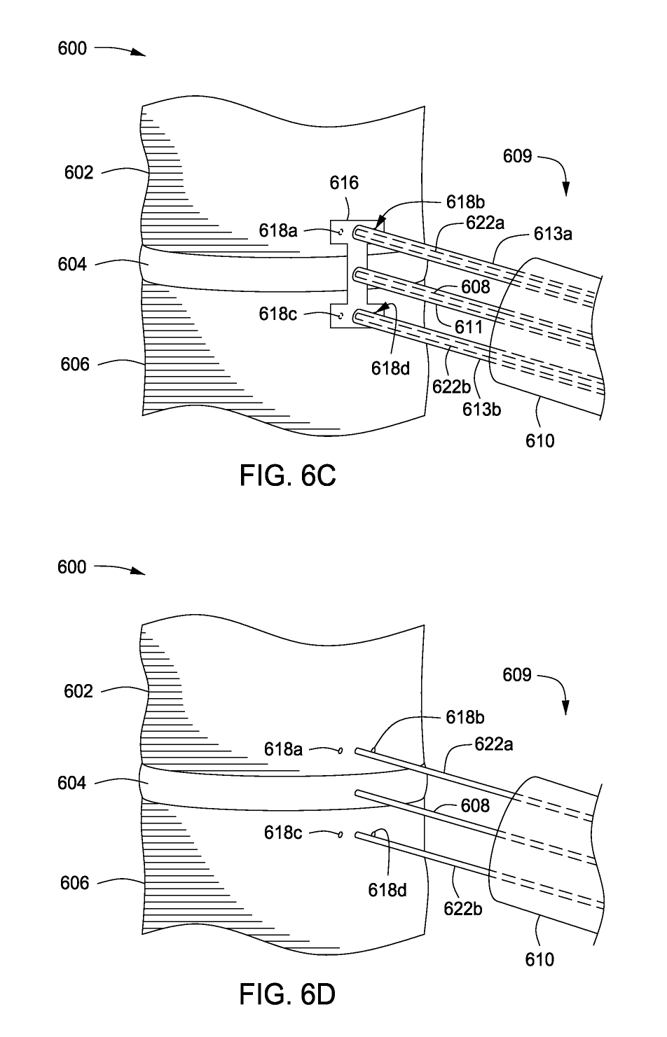

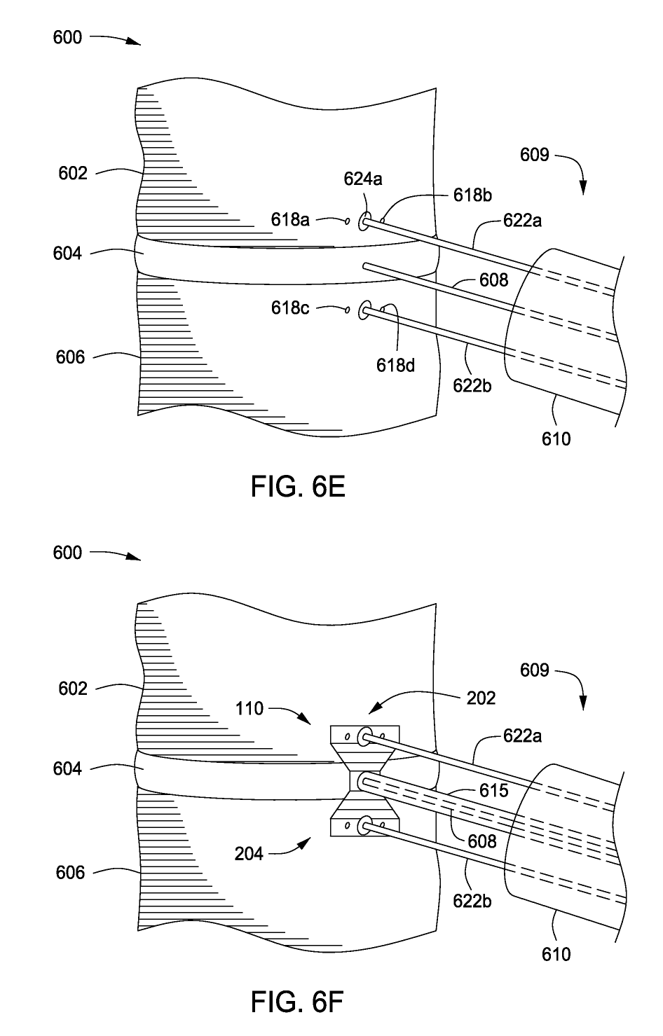

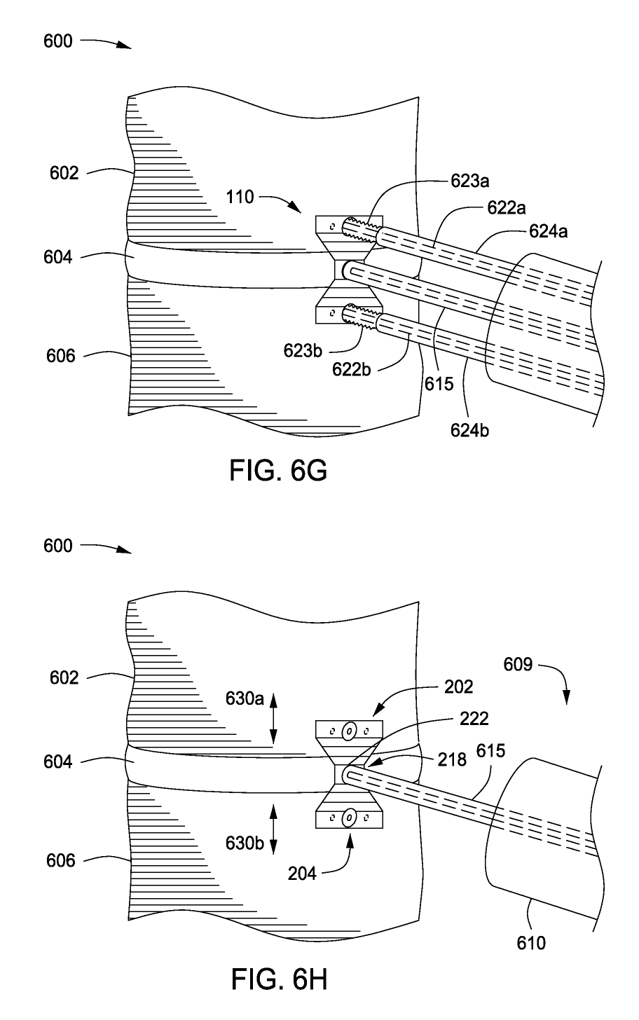

FIG. 5 depicts a flow diagram of a method for compressing or decompressing a spinal disc in accordance with some embodiments of the present invention. FIGS. 6A-H are illustrative perspective views of a segment of a vertebral column during different stages of the method for decompressing a spinal disc depicted in FIG. 5, in accordance with some embodiments of the present invention. To best understand the invention, the reader should refer simultaneously to FIG. 5 and FIGS. 6A-H.

The method 500 begins at step 502 where a disc apparatus 110 is positioned proximate a spinal disc 604. To position the disc apparatus 110, at step 504, a first guide wire 608 is introduced into a spinal disc 604, wherein the spinal disc 604 is located between a first vertebra 602 and second vertebra 606 of a vertebral column 600, as depicted in FIG. 6A. The first guide wire 608 may be any suitable surgical material having mechanical strength sufficient to allow the first guide wire 608 to pierce the spinal disc 604 without bending or breaking. For example, in some embodiments, the first guide wire 608 may comprise a surgical grade stainless steel.

As depicted in FIG. 6B, in some embodiments, a working cannula 609 is positioned over the first guide wire 608. In such embodiments, the working cannula 609 may comprise an inner sleeve 612 and an outer sleeve 610. The inner sleeve 612 comprises a through hole 614 having a diameter slightly larger than that of the outer diameter of the first guide wire 608 to allow for the working cannula 609 to be guided in place when passed over the first guide wire 608. Following the placement of the working cannula 609, the inner sleeve 612 may be removed. The outer sleeve 610 may be left in place and functions as a conduit to allow a user access to the vertebral column 600 to perform the method 500.

Referring back to FIG. 5, next at step 506, two or more features 618 a-d are formed in the first vertebrae 602 and second vertebrae 604, as depicted in FIG. 6C. To form the features 618 a-d, a targeting device 616 having two or more posts (not shown) is passed through the working cannula 609 along the first guide wire 608 and pressed against the first vertebrae 602 and second vertebrae 606 using at least one or more hollow arms (three shown) 611, 613a, 613b.

The two or more posts of the targeting device 616 comprise pointed ends to facilitate breaching the surface of the first vertebrae 602 and second vertebrae 606. In some embodiments, additional pressure is applied to the targeting device 616 to create the features 618 a-d. For example, a blunt object, such as a hammer, is utilized to strike the end of the one or more arms 611, 613a, 613b to cause the two or more posts to breach the surface of the first vertebrae 602 and second vertebrae 606, thus creating the features 618 a-d.

Referring back to FIG. 5, at step 508, in some embodiments, two or more additional guide wires (two shown) 622a, 622b are introduced via the topmost (superior) hollow arm 613a and bottom most (inferior) hollow arm 613b into the first vertebrae 602 and second vertebrae 606, as depicted in FIG. 6C. The targeting device 616 and the one or more hollow arms 611, 613a, 613b are then removed, as depicted in FIG. 6D.

Referring back to FIG. 5, next at step 510, drill holes (two shown) 624a, 624b are created at the locations where the additional guide wires 622a, 622b enter the first vertebrae 602 and second vertebrae 606, as depicted in FIG. 6E. In some embodiments, the drill holes 624a, 624b are formed using a cannulated drill bit. The cannulated drill bit is passed through the working cannula 609 along the additional guide wires 622a, 622b.

Referring back to FIG. 5, next at step 512, the disc apparatus 110, described above with respect to FIGS. 2-5, is passed through the working cannula 609 along the first guide wire 608 and the two additional guide wires 622a, 622b, as depicted in FIG. 6F. The disc decompression apparatus 110 is positioned and held in place using a hollow arm 615.

Referring back to FIG. 5, the method then proceeds to step 514, where the first section 202 and the second section 204 of the disc decompression apparatus 110 is coupled to the first vertebrae 602 and the second vertebrae 606, respectively. The first section 202 and the second section 204 of the disc decompression apparatus 110 may be coupled to the first vertebrae 602 and the second vertebrae 606 via any fastener 623a, 623b suitable to facilitate a secure coupling. For example, the fastener 623a, 623b may be a bolt, anchor, screw, or the like. Two working arms 624a, 624b are passed through the working cannula 609 along the additional guide wires 622a, 622b to deliver and secure the fastener 623a, 623b, as depicted in FIG. 6G. The two working arms 624a, 624b and the two additional guide wires 622a, 622b are then removed.

Referring back to FIG. 5, next at step 516, the expandable section 218 of disc apparatus 110 is expanded or contracted, as depicted in FIG. 6H. Operation of the expandable section 218 is described above with respect to FIG. 4. In some embodiments, the hollow arm 615 is rotated, rotating the screw 222, driving it in a lateral direction into the expandable section 218. As the screw 222 progresses deeper into the expandable section 218, a force is applied simultaneously to the first section 202 and second section 204 in a vertical direction 630a, 630b, increasing the distance between the first section 202 and second section 204, thereby causing the expandable section 218 to expand, thus decompressing the spinal disc 604. Alternatively, in some embodiments, the hollow arm 615 is rotated, rotating the screw 222 out of the expandable section 218. As the screw 222 retreats from the hole 220 of the expandable section 218, the force applied to the first section 202 and second section 204 is decreased, thereby causing the expandable section 218 to contract, thus compressing the spinal disc 604.

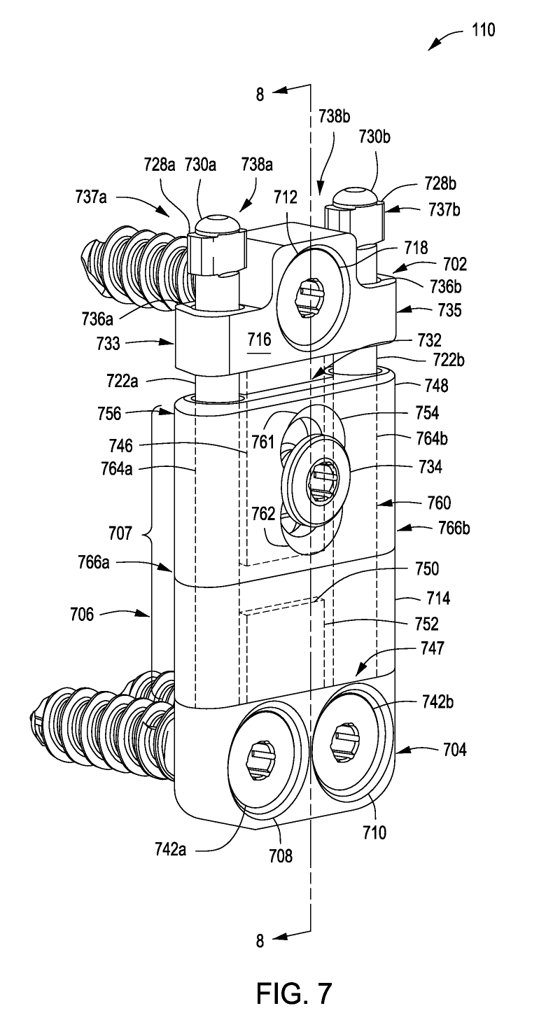

Although FIGS. 1-6 depict the disc apparatus 110 having certain configurations, it is to be noted that the disc apparatus 110 may be configured in any manner suitable to allow the disc apparatus 110 to compress or decompress a spinal disc. For example, in some embodiments, the disc apparatus 110 may be configured such as depicted in FIG. 7. In such embodiments, the disc apparatus 110 may generally comprise a first section 702, second section 704 coupled to an expandable section 706.

The first section 702, second section 704 and expandable section 706 may comprise any material suitable to provide sufficient mechanical strength and stiffness to the disc apparatus 110 while having a low overall weight. For example, the first section 702, second section 704 and expandable section 706 may comprise a metal, plastic, ceramic, or the like. In some embodiments, the metal may be at least one of titanium, steel, aluminum, alloys thereof, or the like. In some embodiments, the material comprises a tensile strength sufficient to resist permanent deformation under the stresses applied thereon resulting from the natural movement of the vertebral column. In some embodiments, the tensile strength may be between about 200 to about 1000 MPa. For example, in embodiments where the material comprises a stainless steel, the tensile strength may be between about 200 to about 225 MPa. Alternatively, in embodiments where the material comprises an aluminum alloy, the tensile strength may be between about 800 to about 925 MPa. The material may also comprise one or more other beneficial properties, for example, resistance to corrosion, pitting, abrasion, chemical degradation, and the like.

In some embodiments, the first section 702 may generally comprise a body 716 defining one or more through holes (one shown) 712 configured to receive a fastener 718 to facilitate coupling the first section 702 to a vertebra. The body 716 may comprise any shape suitable to provide an adequate surface area to facilitate a stable coupling of the first section 702 to a vertebra. For example, in some embodiments, the body 716 may be square, rectangular, triangular, circular, or the like. In some embodiments, the body 716 may comprise an irregular shape, for example a T-shape, such as depicted in FIG. 7.

The fastener 718 may be any fastener suitable to provide a secure and permanent coupling. For example, the fastener may be a screw, bolt, anchor, or the like. In embodiments where the fastener 718 is threaded (e.g. screw or bolt) the through hole 712 may comprise threads configured to interface with threads of the fastener 718 used. The fastener 718 may be any length suitable to provide a secure coupling, while not damaging the structural integrity of the vertebrae. For example, in some embodiments, the fastener 718 may be between about 2.0 to about 4.0 cm long, or in some embodiments, about 2.5 to about 3.5 cm long.

In some embodiments, the first section 702 further defines a first through hole 736a disposed proximate a first side 733 of the body 716 and a second through hole 736b disposed proximate the second side 735 of the body 716. Each of the first through hole 736a and second through hole 736b may be configured to interface with a respective guide post 722a, 722b coupled to the second section 704, thereby movably coupling the first section 702 to the second section 704. In some embodiments, each of the guide posts 722a, 722b may comprise an end cap 737a, 737b disposed proximate an end 738a, 738b of the guide posts 722a, 722b configured to prevent the first section 702 from moving past the end 738a, 738b of the guide posts 722a, 722b. In some embodiments, the end cap 736a, 736b may comprise a stopper 728a, 728b coupled to the end 738a, 738b of the guide posts 722a, 722b via a fastener (e.g., a screw, bolt, or the like) 730a, 730b.

In some embodiments, the first section 702 further comprises a tab 746 coupled to and extending laterally outward from the body 716. The tab 746 (described below) is configured to interface with a recess 732 formed in the expandable section 706 and facilitates coupling the first section 702 to the expandable section 706.

In some embodiments, the second section 704 generally comprises a body 740 defining one or more through holes (two shown) 708, 710 configured to each receive a fastener 742a, 742b to facilitate coupling the first section 702 to a vertebra and two or more (two shown) guide posts 722a, 722b coupled to the body 740 to facilitate movably coupling the first section 702 to the second section 704.

The body 740 may comprise any shape suitable to provide an adequate surface area to facilitate a stable coupling of the section 704 to a vertebra. For example, in some embodiments, the body 740 may be square, triangular, circular, or the like. In some embodiments, for example, as depicted in FIG. 7, the body 740 may be rectangular. The fasteners 742a, 742b may be any type of fastener suitable to provide a secure and permanent coupling, for example such as the fastener 718, described above. In some embodiments, the fasteners 742a, 742b may be the same type and/or comprise a similar length, or in some embodiments, may be a different type and/or comprise a different length as the fastener 718. In some embodiments, for example, where the fasteners 742a, 742b are threaded, the through holes (two shown) 708, 710 may comprise threads configured to interface with threads of the fasteners 742a, 742b used.

The two or more guide posts 722a, 722b may comprise any material suitable to provide sufficient mechanical strength and stiffness to adequately couple the first section 702 to the second section 704 while having a low overall weight. For example, the two or more guide posts 722a, 722b may comprise a metal, plastic, ceramic, or the like. In embodiments where the two or more guide posts 722a, 722b comprise a metal, the metal may be at least one of titanium, steel, aluminum, alloys thereof, or the like. In some embodiments, the two or more guide posts 722a, 722b may be coupled to a first end 746 of the body 740 via any means suitable to provide a permanent coupling, for example via welding. Alternatively, in some embodiments the two or more guide posts 722a, 722b and the second section 704 may be fabricated from a single piece of material.

In some embodiments, the second section 704 may further comprise an insert 750 coupled to the first end 747 of the body 740 and configured to interface with a slot 752 disposed within the expandable section 706. When present, the insert 750 facilitates a secure coupling of the second section 704 to the expandable section 706 and provides overall strength and stability to the disc apparatus 110. In some embodiments, the insert 750 may be coupled to a first end 747 of the body 740 via any means suitable to provide a permanent coupling, for example via welding. Alternatively, in some embodiments the insert 750 and the second section 704 may be fabricated from a single piece of material.

The expandable section 706 is coupled to the second section 704 and movably couples the first section 702 to the second section 704. In some embodiments, the expandable section 706 generally comprises a body 707 having an expansion slot 754 formed in a front surface 760 of the body 707 and extending through to a back surface 756 of the body 707. In some embodiments, the body 707 comprises a first portion 748 and second portion 714, wherein the expansion slot 754 is disposed in the first portion 748 and the slot 752 is disposed in the second portion 714. In some embodiments, the first portion 748 and second portion 714 may be fabricated from a single piece of material. Alternatively, in some embodiments, the first portion 748 and second portion 714 may be separate parts coupled together to form the body 707. In some embodiments, the body 707 defines two or more (two shown) through holes 764a, 764b disposed proximate a first side 766a and second side 766b of the body 707, respectively, and configured to interface with the guide posts 722a, 722b.

In some embodiments, the expansion slot 754 is configured to interface with a screw 734 to facilitate coupling the expandable section 706 to the tab 746 of the first section 702. The expansion slot 754 may comprise any shape suitable to allow the screw 734 to be moved and secured in various positions along the first portion 748 of the expandable section 706. For example, in some embodiments, the expansion slot 754 may comprise an oval shape. Alternatively, in some embodiments, the expansion slot 754 may comprise two or more (two shown) adjacent circular sections 761, 762 configured to hold the screw 734 in a static position when tightened.

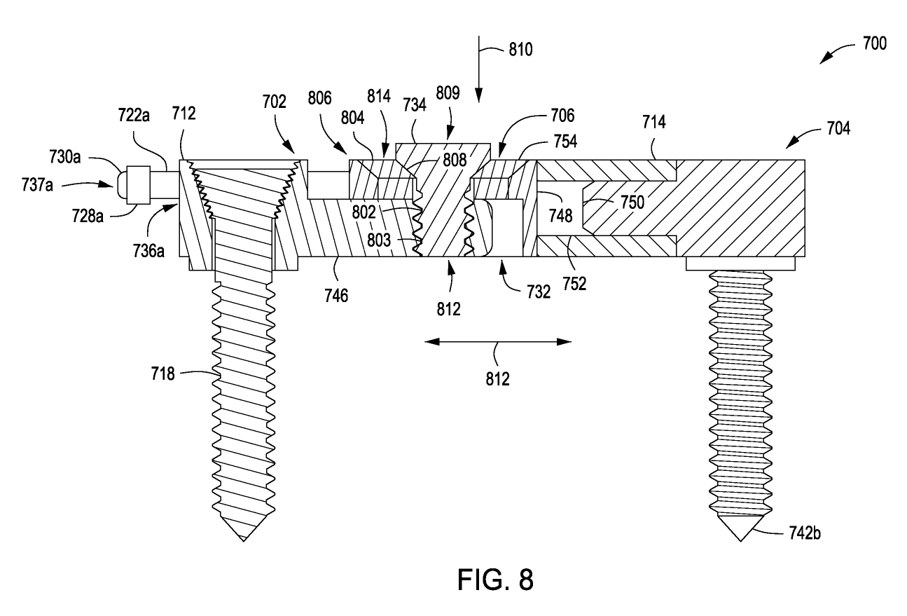

Referring to FIG. 8, in some embodiments, the expansion slot 754 may comprise a top section 806 having a tapered profile 804 configured to interface with a sloped profile 808 of the screw 734. When present, the tapered profile 804 allows the screw 734 to be driven into the expansion slot 754 such that the head 809 of the screw 734 rests flush, or slightly recessed, with respect to an outer surface 814 of the expandable section 706. In addition, the tapered profile 804 prevents lateral slipping of the screw 734 when secured. In some embodiments, the screw 734 may comprise threads 802 configured to interface with threads 803 formed in a through hole 812 of the tab 746.

In operation, the disc apparatus 110 is coupled to a vertebral column via coupling the first section 702 and the second section 704 to a respective first and second adjacent vertebra (for example as described above). The expandable section 706 is then adjusted in a lateral direction (arrow 812) to apply a desired amount of compression or decompression to a vertebral disc between the first and second vertebra. When the desired amount of compression or decompression is achieved the screw 734 is rotated, driving the screw 734 inward (arrow 810) towards the disc apparatus 110 via the threads 803 of through hole 812, thereby securing the tab 744 within the recess 732, thus causing the first section 702 and second section 704 to remain in a static position with respect with one another.

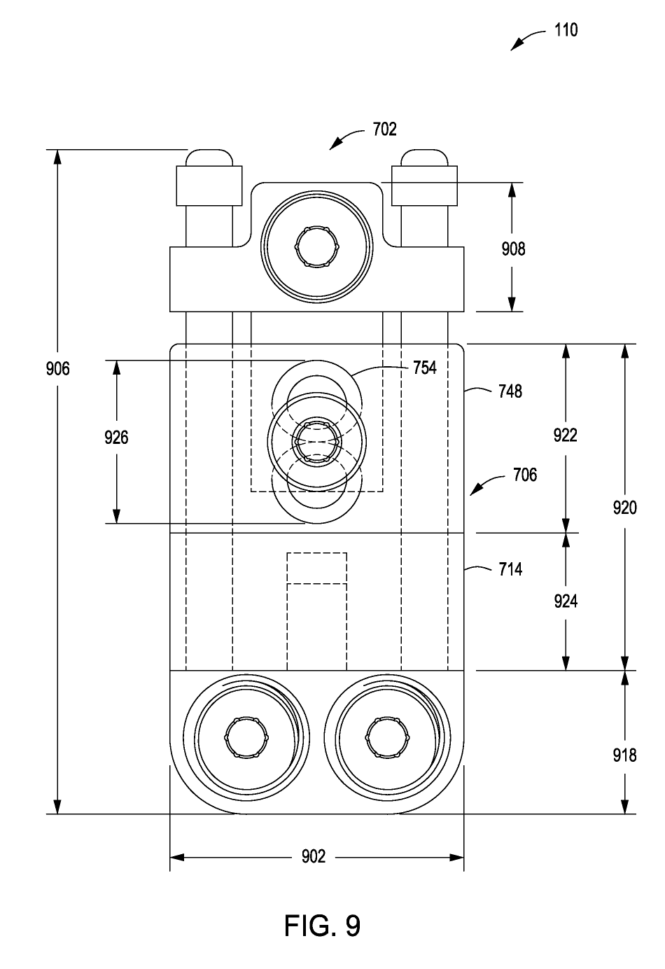

Referring to FIG. 9, in some embodiments, the disc apparatus 110 may comprise any dimensions sufficient to securely couple each of the first section 702 and second section 704 to a respective vertebra and provide support to the vertebral column and facilitate decompression of a damaged spinal disc while not impeding movement or only partially restricting movement thereof. Additionally, the dimensions of the disc apparatus 110 may be varied to accommodate the various sizes of vertebrae with respect positions of the spinal column (e.g., the lumber, thoracic and cervical regions).

In addition, although the components of the disc apparatus 110 are shown having a certain proportion with respect to one another, the dimensions of each of the components may be varied to securely couple each of the first section 702 and second section 704 to a respective vertebra and provide support to the vertebral column and facilitate decompression of a damaged spinal disc while not impeding movement or only partially restricting movement thereof. For example, one or more of the overall height (arrow 906) of the disc apparatus 110, the width (arrow 902) of the disc apparatus 110, the height (arrow 908) of the first section 702, the height (arrow 918) of the second section 704, the overall height (arrow 920) of the expandable section 706, the height (arrow 922) of the first portion 748 of the expandable section 706 or the height (arrow 924) of the expandable section 706 may be varied to securely couple each of the first section 702 and second section 704 to a respective vertebra and provide support to the vertebral column and facilitate decompression of a damaged spinal disc while not impeding movement or only partially restricting movement thereof.

While the foregoing is directed to embodiments of the present invention, other and further embodiments of the invention may be devised without departing from the basic scope thereof, and the scope thereof is determined by the claims that follow.

1. An apparatus for compressing or decompressing a spinal disc, comprising:
a first section and a second section, wherein the first section defines a through hole configured to receive a fastener for coupling the first section to a first vertebrae, and wherein the second section defines a through hole configured to receive a fastener for coupling the second section to a second vertebrae; and
an expandable section coupled to the first section and the second section for adjusting a distance between the first and second section.

2. The apparatus of claim 1, wherein the fastener is at least one of a bolt, anchor, or threaded screw.

3. The apparatus of claim 1, wherein the expandable section further comprises:
an expansion slot formed therein, wherein the expansion slot is formed in a front surface of the expandable section and extends through to a back surface of the expandable section and wherein the expansion slot is configured to receive a screw.

4. The apparatus of claim 3, wherein the expansion slot comprises two or more adjacent circular sections, each of the adjacent circular sections configured to hold the screw in a static position when tightened.

5. The apparatus of claim 3, wherein the expansion slot comprises a top section having a tapered profile configured to interface with a sloped profile of the screw.

6. The apparatus of claim 3, wherein the first section further comprises:
an outwardly extending tab configured to interface with a recess formed in the back surface of the expandable section, wherein the outwardly extending tab comprises a through hole having threads configured to interface with threads of the screw, and wherein the screw movably couples the first section to the expandable section.

7. The apparatus of claim 3, wherein the expandable section further comprises
a first portion having the expansion slot formed therein; and
a second portion having a slot formed therein.

8. The apparatus of claim 7, wherein the second section comprises an outwardly extending tab configured to interface with the slot for coupling the second section to the expandable section.

9. The apparatus of claim 1, wherein the second section defines an additional through hole configured to receive a fastener for coupling the second section to the second vertebrae.

10. The apparatus of claim 1, wherein the second section comprises at least two guide posts coupled to a top surface of the second section.

11. The apparatus of claim 10, wherein the expandable section comprises at least two through holes configured to interface with the at least two guide posts.

12. The apparatus of claim 10, wherein the first section comprises at least two through holes configured to interface with the at least two guide posts, and wherein the first section is movably coupled to the at least two guide posts.

13. The apparatus of claim 1, wherein the first, second section and expandable section comprise at least one of a metal, ceramic, or plastic.

14. The apparatus of claim 13, wherein the metal is titanium, vanadium, steel, or alloys thereof.

15. The apparatus of claim 13, wherein the metal comprises a tensile strength of about 200 to about 1000 MPa.
